(19) United States
(12) Patent Application Publication
(10) Pub. No.: US 2022/0291213 A1
(43) Pub. Date: Sep. 15, 2022

(54) LATERAL FLOW DEVICES AND METHODS

(71) Applicant: SALUS DISCOVERY, LLC, Monona, WI (US)

(72) Inventor: David J. Guckenberger, Madison, WI (US)

(21) Appl. No.: 17/711,222

(22) Filed: Apr. 1, 2022

Related U.S. Application Data
(63) Continuation of application No. 16/624,099, filed on Dec. 18, 2019.
(60) Provisional application No. 62/616,775, filed on Jan. 12, 2018.
(60) Provisional application No. 62/522,495, filed on Jun. 20, 2017.

(51) Int. Cl. G01N 33/558; G01N 33/53; G01N 33/543; G01N 35/00

(52) U.S. Cl. CPC G01N 33/558; G01N 33/5304; G01N 33/54326; G01N 35/0098

(57) ABSTRACT

Provided herein are devices, systems, and methods for purification of sample components. In particular, provided herein are lateral flow devices, systems, and methods that utilize flow control of sample handling.

### CROSS REFERENCE TO RELATED APPLICATION

This application is a continuation of U.S. application Ser. No. 16/624,099, filed Dec. 18, 2019, which is a 35 U.S.C. § 371 of PCT International Application No. PCT/US2018/038494, filed Jun. 20, 2018, which claims priority to and the benefit of U.S. Provisional Application No. 62/522,495, filed Jun. 20, 2017, and U.S. Provisional Application No. 62/616,775, filed Jan. 12, 2018, the contents of which are hereby incorporated by reference in their entireties.

### FIELD

Provided herein are devices, systems, and methods for purification of sample components. In particular, provided herein are lateral flow devices, systems, and methods that utilize flow control of sample handling.

### BACKGROUND

There is a great need for cost-effective, easy to use systems, methods, and devices for analyzing biological samples. Many commercially available systems cost tens to hundreds of thousands of dollars and have many moving parts which make them prone to failure. Because of the cost and complexity of such systems, their use has generally been limited to clinical laboratories which have the personnel and services needed to support their operation and maintenance.

One class of fully integrated automated analyzers, represented by the Abbott Architect, Siemens Centaur, Roche Elecsys, and others, perform immunoassays. Another class of modular analyzers, represented by the Abbott m2000, Roche COBAS, bioMérieux NucliSENS and others, perform nucleic acid assays. Much of the complexity of these systems is a result of separation steps involved in processing the assays.

Modular systems are also frequently used in research laboratories. Immunoassay separations may be performed by plate washers such as Titertek MAP-C2, BioTek ELx50, Tecan PW 96/384 and others. Nucleic acid separations are performed by systems such as the Applied Biosystems PRISM™ 6100, Invitrogen iPrep, Thermo Scientific KingFisher, Promega Maxwell, and others.

Existing assay systems and methods are complex, expensive and not suitable for use in many settings, especially in the developing world. Additional systems and methods are needed.

### SUMMARY

Provided herein are devices, systems, and methods for purification of sample components. In particular, provided herein are lateral flow devices, systems, and methods that utilize flow control of sample handling.

For example, in some embodiments, provided herein is a lateral flow assay device, comprising one or more of: a) a sample concentration component; b) a sample loading component; and c) a test component comprising a test region and/or a control region. In some embodiments, the sample concentration component is or is not in operable communication with the sample loading component. In some embodiments, the test component is a membrane. The present disclosure is not limited to particular sample concentration components. Examples include, but are not limited to, a magnet, a cleavable molecule, a porous membrane, and a reservoir.

In some embodiments, the present disclosure provides a lateral flow assay device, comprising: a sample loading region in operable communication with a concentration component; and a membrane comprising a test region and a control region. In some embodiments, the concentration component is a magnet. In some embodiments, the magnet is a permanent or semi-permanent magnet. In some embodiments, the magnet is a disk magnet or electromagnet. In some embodiments, the device further comprises a magnet transport component configured to move said magnet into and out of operable communication with the sample loading region. In some embodiments, the sample loading region comprises a plurality of magnetic particles (e.g., comprising a reagent that specifically binds to an analyte). In some embodiments, the reagent is antibody, a lectin, a carbohydrate, a dye, biotin, or strepavidin. In some embodiments, the device further comprises a housing. In some embodiments, the housing is a fluid collection container (e.g., cup) with the device integrated into the lid of the cup. In some embodiments, the concentration component is inside or outside of the housing. In some embodiments, the device or housing comprises a plurality of such lateral flow assays. In some embodiments, the sample loading region is a section of the membrane. In some embodiments, the sample loading region is not a section of the membrane (e.g., is a funnel or tube or other delivery component). In some embodiments, the device further comprises a fluid handling component. In some embodiments, the fluid handling component comprises a sample pad in operable communication with the sample loading component and a plurality of absorbent pads. In some embodiments, the absorbent pads are located upstream and/or downstream (e.g., relative to the flow of sample) to the sample pad. In some embodiments, the fluid handling component further comprises a physical, chemical, or other separation component (e.g., removable barrier) that separates the fluid handling component and the sample loading component from an test component portion of the device (e.g., membrane).

Additional embodiments provide a lateral flow assay device, comprising: a sample loading region in operable communication with a sample concentration component (e.g., magnet); a membrane comprising a test region and/or a control region; and a fluid handling component comprising a sample pad in operably communication with the sample loading region and a plurality of absorbent pads.

Certain embodiments provide a sample collection device, comprising: a sample loading region in operable communication with a sample concentration component (e.g., magnet); and a fluid handling component comprising a sample pad in operably communication with the sample loading region and a plurality of absorbent pads. In some embodiments, the sample collection component is configured to integrate with a membrane comprising a test and/or control region. In some embodiments, the sample collection device is integrated into a lid for a sample collection container.

In further embodiments, the present disclosure provides a system or kit, comprising: a) a device as described herein; and b) a wash buffer.

Yet other embodiments provide a method of detecting an analyte, comprising: a) introducing a sample into the sample loading region of a device as described herein with the sample concentration component (e.g., magnet) in operable communication with the sample loading region under conditions such that the magnet retains said magnetic particles in the sample loading region and the analyte binds to the magnetic particles; and b) releasing concentrated sample from the concentration component (e.g., by removing the magnet from operable communication with the sample loading region) and optionally adding the wash buffer to the sample loading region under conditions such that the magnetic particles flow onto the membrane and contact the test region and the control region. In some embodiments, the magnetic field is applied to the sample loading region for sufficient time to concentrate more of the target analytes from the sample on the magnetic particles as compared to a similar device that does not retain the particles in the sample loading region. In some embodiments, the magnet is moved using a method selected from switching an electromagnet on and off or physically moving the magnet into and away from proximity to the sample loading region.

In some embodiments, the analyte is, for example, a protein, a peptide, a carbohydrate, a lipid, a nucleic acid, a hormone, a metabolite, or a microorganism specific marker. In some embodiments, the sample is, for example, a blood sample, a blood product sample (e.g., serum, plasma), a urine sample, a food sample, sputum, stool, water, semen, milk, or a saliva sample. In some embodiments, the method further comprises the step of contacting the sample with the sample pad under conditions such that fluid from the sample (e.g., excess fluid) flows to the absorbent pads. In some embodiments, the method further comprises the step of removing the barrier after the fluid flows to the absorbent pads.

Additional embodiments are described herein.

### BRIEF DESCRIPTION OF THE DRAWINGS

FIG. 1A-1E show exemplary devices comprising sample concentration components.

FIG. 2A-2C show exemplary device configurations.

FIG. 3 shows exemplary fluid handling components.

FIG. 4A and FIG. 4B shows exemplary fluid handling components.

FIG. 5 shows an exemplary fluid collection cup comprising an integrated device of embodiments of the present disclosure.

FIG. 6 shows a detail view of the lid of collection cup of FIG. 5.

FIG. 7 shows a detail view of an alternative embodiment of a lid of a collection cup.

FIG. 8 shows a schematic of a fluid collection cup with integrated device in use.

FIG. 9 shows an exemplary device of embodiments of the present disclosure.

FIG. 10 shows an exemplary device of embodiments of the present disclosure.

FIG. 11 shows an exemplary device of embodiments of the present disclosure.

FIG. 12 shows an exemplary device of embodiments of the present disclosure.

FIG. 13 shows an exemplary device of embodiments of the present disclosure.

FIG. 14 shows an exemplary device of embodiments of the present disclosure.

### DEFINITIONS

To facilitate an understanding of the present disclosure, a number of terms and phrases are defined below:

The term “sample” is used in its broadest sense. On the one hand it is meant to include a specimen or culture. On the other hand, it is meant to include both biological and environmental samples. A sample may include a specimen of synthetic origin. Biological samples may be obtained from animals (including humans) and encompass fluids (e.g., urine, blood, blood products, sputum, saliva, etc.), solids, tissues, and gases. Biological samples include blood products, such as plasma, serum and the like. In some embodiments, samples contain or are suspected of containing a microorganism (e.g., a pathogenic or disease-causing microorganism).

As used herein, the term “cell” refers to any eukaryotic or prokaryotic cell (e.g., bacterial cells such as E. coli, yeast cells, mammalian cells, avian cells, amphibian cells, plant cells, fish cells, and insect cells), whether located in vitro or in vivo.

“Analyte,” as used herein, is the substance to be detected which may be present in the test sample, including, biological molecules of interest, small molecules, pathogens, and the like. The analyte can include a cell, a microorganism, a protein, a polypeptide, an amino acid, a nucleotide target and the like. The analyte can be soluble in a body fluid such as blood, blood plasma or serum, urine or the like. The analyte can be in a tissue, either on a cell surface or within a cell. The analyte can be on or in a cell dispersed in a body fluid such as blood, urine, breast aspirate, or obtained as a biopsy sample.

A “capture reagent,” as used herein, refers to a labeled or unlabeled specific binding member, which is specific either for the analyte as in a sandwich assay, for the indicator reagent or analyte as in a competitive assay, or for an ancillary specific binding member, which itself is specific for the analyte, as in an indirect assay. The capture reagent can be directly or indirectly bound to a solid phase material before the performance of the assay or during the performance of the assay, thereby enabling the separation of immobilized complexes from the test sample.

The “indicator reagent” comprises a “signal-generating compound” (“label”) which is capable of generating and generates a measurable signal detectable by external means. In some embodiments, the indicator reagent is conjugated (“attached”) to a specific binding member. In addition to being an antibody member of a specific binding pair, the indicator reagent also can be a member of any specific binding pair, including either hapten-anti-hapten systems such as biotin or anti-biotin, avidin or biotin, a carbohydrate or a lectin, a complementary nucleotide sequence, an effector or a receptor molecule, an enzyme cofactor and an enzyme, an enzyme inhibitor or an enzyme and the like. An immunoreactive specific binding member can be an antibody, an antigen, or an antibody/antigen complex that is capable of binding either to the polypeptide of interest as in a sandwich assay, to the capture reagent as in a competitive assay, or to the ancillary specific binding member as in an indirect assay. When describing probes and probe assays, the term “reporter molecule” may be used. A reporter molecule comprises a signal generating compound as described hereinabove conjugated to a specific binding member of a specific binding pair, such as carbazole or adamantane.

The various “signal-generating compounds” (labels) contemplated include chromagens, catalysts such as enzymes, luminescent compounds such as fluorescein and rhodamine, chemiluminescent compounds such as dioxetanes, acridiniums, phenanthridiniums and luminol, radioactive elements and direct visual labels. Examples of enzymes include alkaline phosphatase, horseradish peroxidase, beta-galactosidase and the like. The selection of a particular label is not critical, but it should be capable of producing a signal either by itself or in conjunction with one or more additional substances.

“Solid phases” (“solid supports”) are known to those in the art and include the walls of wells of a reaction tray, test tubes, polystyrene beads, magnetic or non-magnetic beads, nitrocellulose strips or other lateral flow strips, membranes, microparticles such as latex particles, and others. The “solid phase” is not critical and can be selected by one skilled in the art. Thus, latex particles, microparticles, magnetic or non-magnetic beads, membranes, plastic tubes, walls of microtiter wells, glass or silicon chips, are all suitable examples. It is contemplated and within the scope of the present invention that the solid phase also can comprise any suitable porous material.

As used herein, the terms “detect”, “detecting”, or “detection” may describe either the general act of discovering or discerning or the specific observation of a detectably labeled composition.

The term “antibody” herein is used in the broadest sense and encompasses various antibody structures, including but not limited to monoclonal antibodies, polyclonal antibodies, multispecific antibodies (e.g. bispecific antibodies), and antibody fragments so long as they exhibit the desired antigen-binding activity. Also included are antibody fragments having an Fc region, and fusion proteins that comprise a region equivalent to the Fc region of an immunoglobulin.

An “antibody fragment” refers to a molecule other than an intact antibody that comprises a portion of an intact antibody that binds the antigen to which the intact antibody binds. Examples of antibody fragments include but are not limited to Fv, Fab, Fab′, Fab′-SH, F(ab′)2, single-chain antibody molecules (e.g. scFv), diabodies, and multispecific antibodies formed from antibody fragments.

### DETAILED DESCRIPTION

Provided herein are devices, systems, and methods for purification of sample components. In particular, provided herein are lateral flow devices, systems, and methods that utilize flow control of sample handling.

Provided herein are devices, systems, and methods that integrate sample preparation/analyte concentration into lateral flow assays (LFA). In some embodiments, analyte is concentrated in a concentration component. Illustrative embodiments are described below using magnetic particles and magnetic fields. However, it should be understood that other concentration components may be employed. For example, in some embodiments, cleavable molecules fixed in the concentration component are used to capture analyte, and cleavage is initiated to release the concentrated analyte after the desired amount of sample is contacted with the concentration component, permitting flow of analyte to a detection component.

In some embodiments, traditional analyte-specific particles (e.g., microparticles or nanoparticles) (e.g., gold, latex, or carbon) are replaced with analyte-specific magnetically responsive particles. Magnetic nanoparticles are immobilized while in the presence of a magnetic field but free to flow once the magnetic field is removed. Using this technique, analytes (from the sample) are concentrated on the beads, increasing the sensitivity of LFAs, lowering limits of detection, and enabling analysis of a wider range of samples under a wider range of conditions.

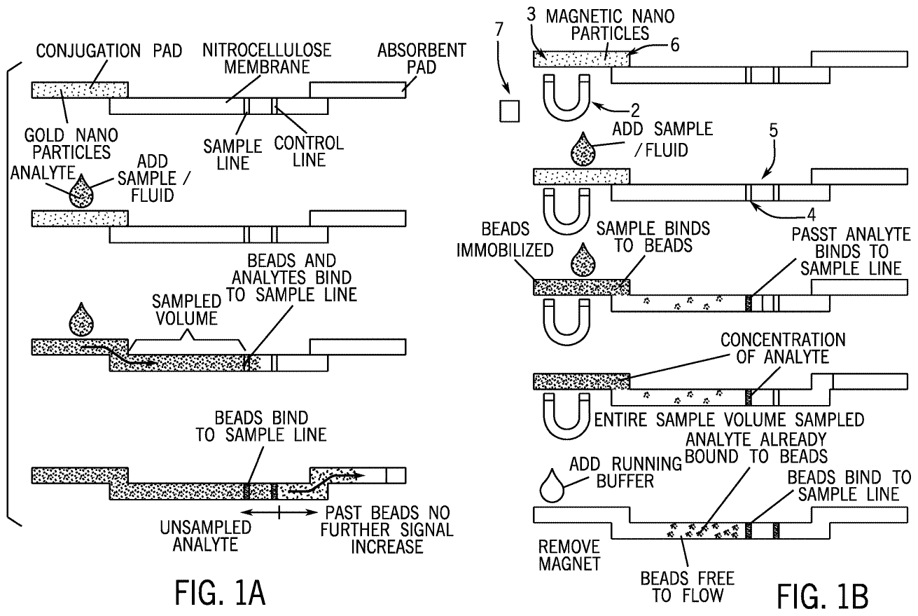

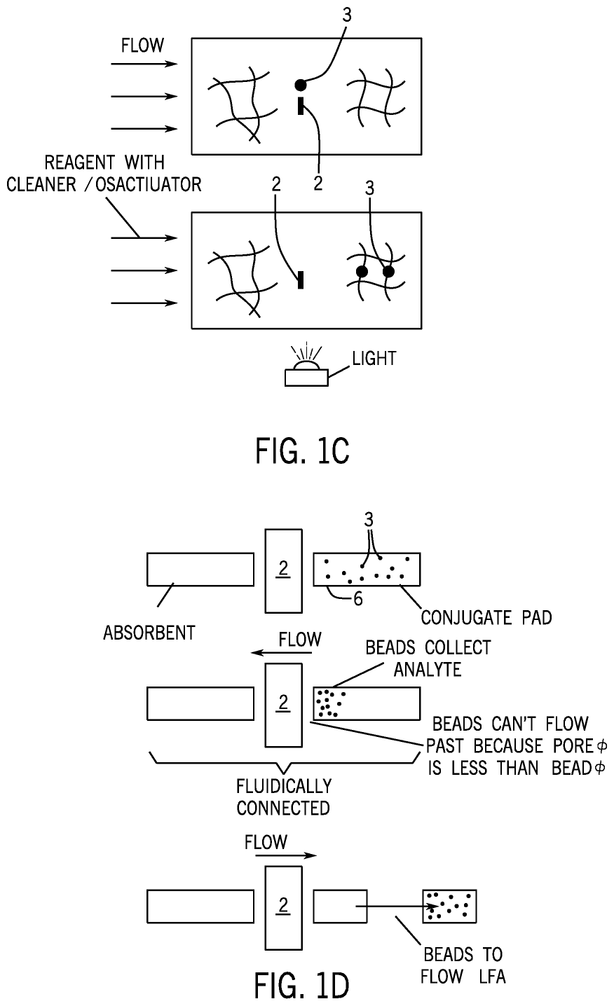

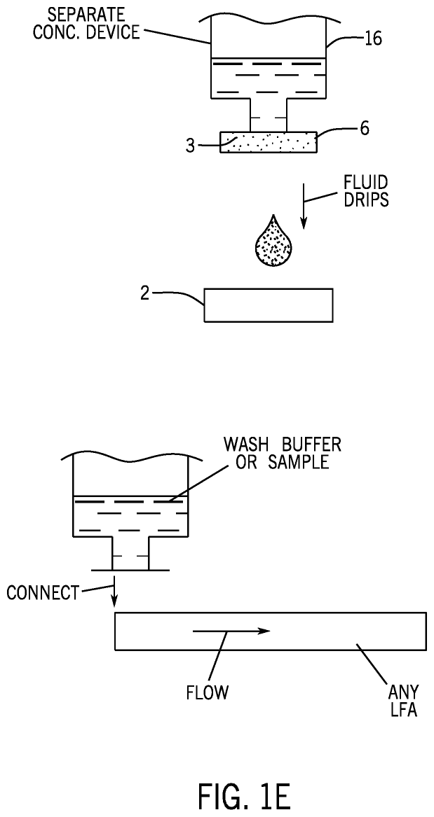

FIG. 1A shows a typical LFA. LFAs are comprised of a series of absorbent pads and membranes that are connected via direct contact. The conjugate pad contains nanoparticles (e.g., latex, gold, or carbon) that are pre-conjugated with an anti-body (e.g., anti-XA) to a specific antigen (e.g., antigen X) of interest. As sample flows through the conjugate pad, the beads are suspended in the fluid, flow with the fluid, and bind to the target analyte. The fluid with the nanoparticles then flows into a nitrocellulose membrane. The nitrocellulose membrane contains two lines of antibody. The first line is an antibody that is specific to antigen X, but typically different than anti-XA (e.g., anti-XB). The second line is a line of control antibody (e.g., anti-Y) specifically used as a confirmation that the test functioned properly. As antigen X flows past the sample line, it binds to anti-XB. Any nanoparticles (which contain anti-XA) that follow antigen X bind to antigen X and hence bind to the sample line, which creates the visual signal (e.g., the colored line). Similarly, any antigen X that is already bound to the nanoparticle (via anti-XA) binds to the sample line. Through this binding process the signal at the line continuously increases. However, once all of the nanoparticles pass the sample line, the signal is no longer able to increase.

Unfortunately, with traditional lateral flow assays, the majority of the beads flow near the fluid front. As a result, only a fraction of the sample volume is actually sampled. That is, only sample that flow with or in front of the beads is sampled. Any sample that follows the beads does not contribute to the signal. For this reason, typical lateral flow assays have limited sensitivity and are often only compatible with samples that contain high concentrations of antigen (such as pregnancy samples).

One option to concentrate the antigen is to use sample preparation technologies, which are capable of concentrating the sample several fold. The challenge with sample prep technologies is that they add cost, complexity, and time to what is otherwise a very simple assay to perform.

Accordingly, in some embodiments, provided herein are improved LFAs that overcome these problems by providing sample concentration components and/or sample preparation components. In some embodiments, devices provide for a method of concentrating analyte that is directly integrated into the LFA. In some embodiments, LFAs utilizes magnetically responsive nanoparticles, as opposed to the traditional non-magnetic nano-particles. When using magnetic nanoparticles, if a magnet is near the LFA, the nanoparticles immobilize within the fibrous or membranous matrix. Since the nanoparticles are immobilized (and do not flow with the fluid front) sample can be flowed past the beads, allowing analyte to bind to the particles and enabling full use of the sample volume and concentrating the analyte in the beads. Notable, any analyte that passes the beads will bind to the sample line. Once all of the sample has flowed past the beads, the beads are released (de-immobilized) by removing the magnet or deflecting the magnetic field. The nanoparticles are now free to flow. In some embodiments, a running buffer is used to carry the beads towards the sample line, where the analyte and already bound bead bind to create the signal. In some embodiments, all or a portion of the sample volume is used to carry beads towards the sample line.

The present disclosure is not limited to particular sample concentration components. The below disclosure is exemplified with magnetic sample concentration components. However, other suitable sample concentration components find use in the devices, systems, and methods described herein. Examples include, but are not limited to, immobilization of sample with antigen binding partners (e.g., cleavable antibodies), immobilization of sample with a hydrogel, immobilization of sample with a cleavable linker or other molecule (e.g., light cleavable linker), or physically immobilizing sample with a pad, filter, or membrane that only allows beads to flow in one direction. In some embodiments, sample flows through the sample loading compartment and the sample fluid is captured in a container or drain while the sample is captured in the sample concentration component.

Exemplary devices of embodiments of the present disclosure are shown in FIG. 1B-1E. FIG. 1B illustrates an embodiment where the sample concentration component 2 is a magnet. FIG. 1B shows device 1 with sample loading component 6. Sample loading component 6 comprises particles (e.g., magnetic particles) 3. Magnet 2 is in proximity to sample loading component 6 and comprises a magnet transport component 7 configured to move the magnet 2 in and out of operable communication with the magnetic particles 3. Devices further comprise test component comprising test/sample line 4 and/or control line 5.

FIG. 1C shows an embodiment where the sample concentration component 2 is a cleavable linker. The top panel shows a bead or particle 3 immobilized by linker 2. In the bottom panel, a cleavage agent (e.g., light and/or chemical cleavage/deactivator agent) is contacted with the particle, the linker is cleaved, and the sample is able to move into the test/sample line.

FIG. 1D shows an embodiment where the sample concentration component 2 comprises a porous membrane with a pore size smaller than the particles or beads. In FIG. 1D, the components are fluidly connected. In the top panel, a device is shown with sample loading component 6 with particles 3. In the middle panel, sample concentration is shown. Fluid flow is away from the sample/test line. Beads cannot flow past the porous membrane 2 and are concentrated on the sample loading component 6. Fluid flows through the membrane 2 and onto an absorbent pad or other fluid collection component. In the bottom panel, sample release is shown. The direction of fluid flow is reversed such that fluid flow is towards the sample/test line. Sample contacted with beads flows to the sample/test line.

FIG. 1E shows an embodiment where the sample concentration component 2 is a separate device not in fluid contact with the test/sample line component. In some embodiments, the sample concentration component 2 comprises a sample reservoir 16. Sample is introduced to the reservoir and travels to a collection region 6 comprising particles 3. After sample is concentrated, the sample concentration component is then placed in fluid communication with the device comprising test/sample line. In some embodiments, fluid flow is used to transfer the particles comprising analyte into the test device.

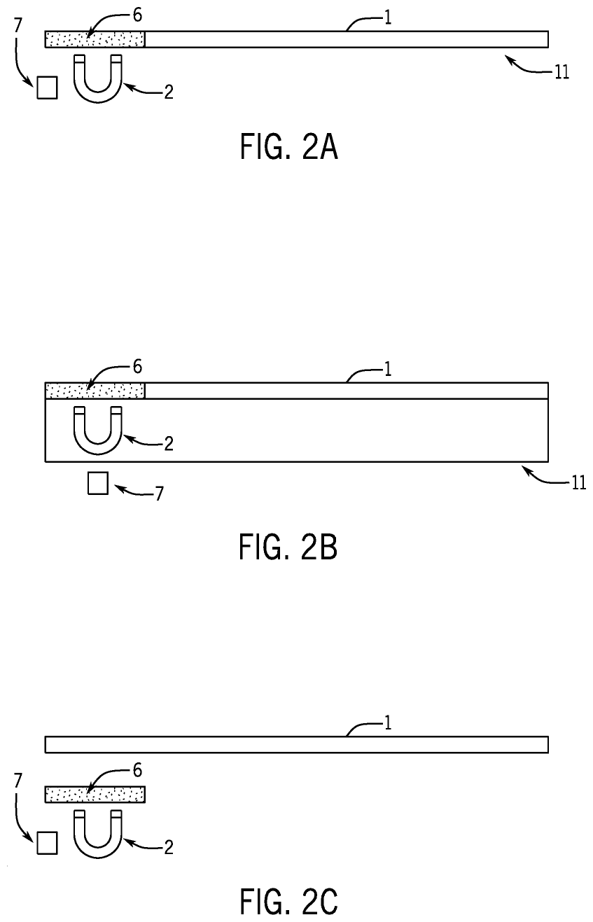

In FIG. 1B and FIGS. 2A and 2B, sample loading component 6 is shown as a compartment adjacent to test/sample line 4. In some embodiments, the test/sample line 4 and/or control line 5 are on a membrane. In some embodiments, sample loading component 6 comprises a section of the same membrane as the test/sample line 4 and/or control line 5. However, the present disclosure is not limited to particular sample loading component configurations. In some embodiments, the sample loading component is a tube or funnel. In some embodiments, the tube or funnel or other sample loading component is in operable communication with a membrane comprising the test and/or control lines during loading. In some embodiments, the sample loading component (e.g., funnel or tube) is not in operable communication during sample loading. For example, in some embodiments, sample is contacted with a sample loading component comprising beads and magnet, allowed to incubate, and then placed in operable communication with the membrane comprising the test and/or control lines. FIG. 2C shows an exemplary embodiment with the sample loading components out of operable communication with the device. In some embodiments, beads are loaded with sample is a component separate from the sample loading component or test line (e.g., conjugate region).

The present disclosure is not limited to particular particles 3. In some embodiments, particles are magnetic or paramagnetic nanoparticles. Such particles commonly include components, a magnetic material, often iron, nickel and cobalt, and a chemical component that has functionality (e.g., binding to an analyte of interest). In some embodiments, magnets are not magnetic (e.g., polymeric or other material).

The present disclosure is not limited to particular magnets. In some embodiments, the magnet is a disk magnet (e.g., commercially available from K&J Magnetics, Inc., Pipersville, Pa.). In some embodiments, the magnet is an electromagnet.

In some embodiments, the magnet transport component 7 is a physical lever, switch, knob, etc. that moves the magnet closer to and away from the sample loading component to operably engage or disengage with the magnetic particles (e.g., to hold them in the sample loading component 6 or release them into the device) or turns an electromagnet on and off. In some embodiments, the magnet transport component is operated by a user. In some embodiments, the magnet transport component is automated or operated by a user. In some embodiments, rather than removing the magnet, the magnetic field is deflected (e.g., an electromagnetic field is deflected).

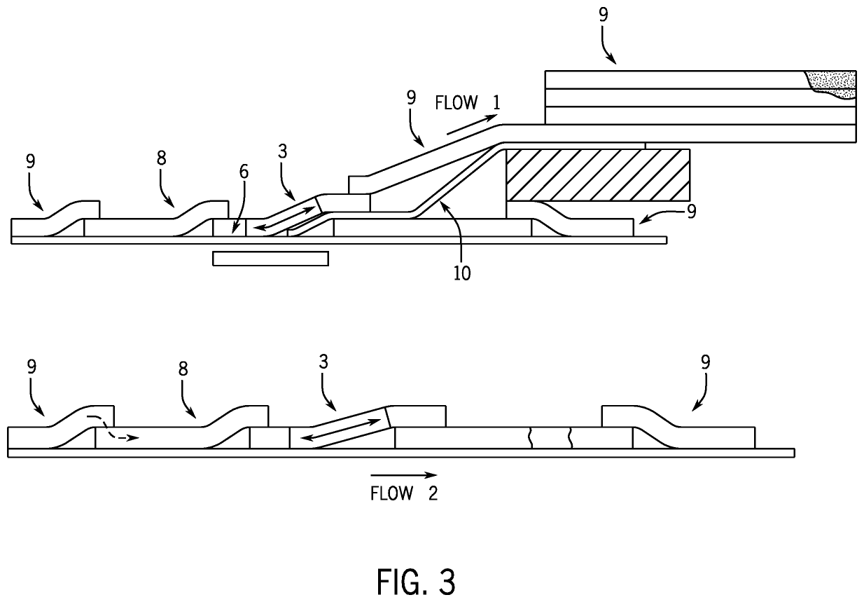

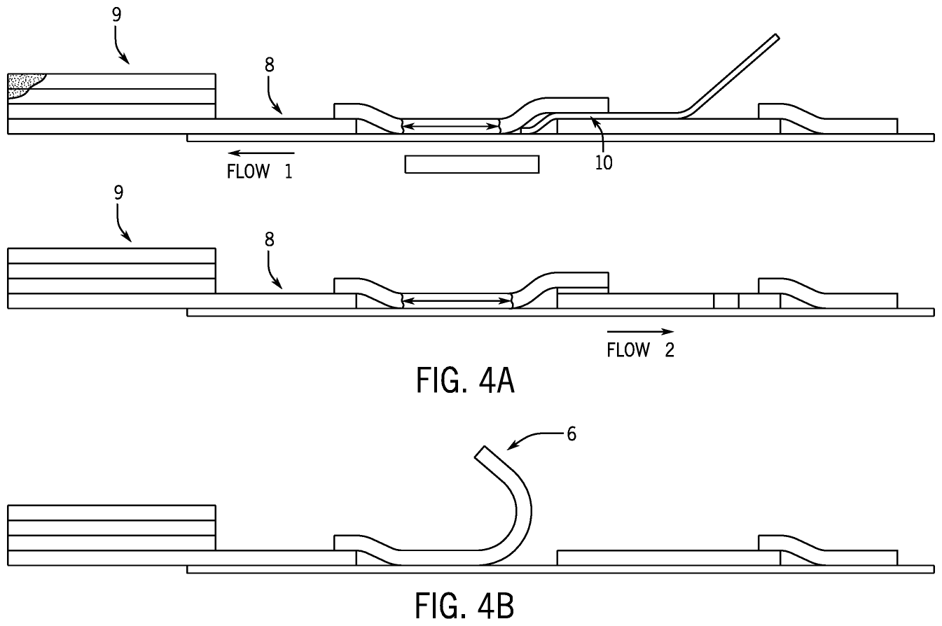

FIGS. 3 and 4 show optional fluid handling components 8-10 that facilitate use of large sample volumes. The fluid handling component captures fluid from the sample. For example, some assays may require a large volume of sample fluid to capture and/or concentrate sufficient amount of analyte for accurate and/or precise detection. Such volumes may overwhelm a traditional flow strip design. Embodiments described herein can accommodate any sample volume. Shown is sample loading pad 8 in operable communication with sample loading component 6 and a plurality (e.g., 1-5) absorbent pads 9. Also shown is removable barrier 10 that prevents sample fluid from entering the membrane containing the detection chemistry. In some embodiments, the device comprises an indicator that indicates (e.g., via color change) when sufficient sample volume has entered the device.

The present disclosure is not limited to particular removable barrier 10. In some embodiments, as shown in FIGS. 3 and 4, the removable barrier is a non-porous film or membrane located between the sample/test line and fluid containment components. In some embodiments, the barrier is an air or fluid gap between the sample/test line and fluid containment components. In some embodiments, the sample loading pad is physically oriented so as to provide a self-contained barrier (e.g., as shown in FIG. 4).

FIG. 3 shows a configuration where a plurality of absorbent pads 9 are placed downstream of the sample loading pad 8 and downstream of the sample loading component 6. The downstream pad 9 is in fluid communication with the sample loading pad and sample loading component but is not in fluid communication with the membrane. In the configuration shown in FIG. 3, sample is added to the sample loading pad 8. Analyte is absorbed on bead in sample loading component 6. Fluid flows downstream and is captured on downstream absorbent pad 9.

FIG. 4 shows an alternative embodiment where a single absorbent pad 9 is located upstream of the sample loading pad 8. Analyte is absorbed on bead in sample loading component 6. Fluid flows upstream and is captured on absorbent pad 9.

In some embodiments, additional running buffer is added after sample fluid, if needed to ensure that sample has contacted beads and all fluid is absorbed in the absorbent pads.

In some embodiments, once the entire sample has contacted the beads and all the fluid is absorbed on the absorbent pads, the removable barrier and bead retention component (e.g., magnet) is removed, allowing the beads to flow to the test/sample line. The removal barrier can be removed using any suitable method. In some embodiments, the device comprises a pull strip or tab external to the device that a user can pull to remove the barrier and allow the assay to proceed. In some embodiments, removal of the barrier is automated (e.g., after an indicator has indicated complete sample loading). In some embodiments, a user physically contacts the sample loading component with the test/sample line by changing the location of the sample loading component to contact the test/sample line. Following removal of the barrier, an additional flow buffer may be added, if needed or desired, to flow the beads to the detection zone. In some embodiments, fluid contained in the absorbent pads may be employed to generate the flow of the beads to the detection zone (e.g., via reduced volume or application of pressure to reverse fluid flow direction, or other suitable mechanism).

In some embodiments, the device comprises a housing (e.g., plastic housing) that encloses the device. In some embodiments, the sample collection component (e.g., magnet) is located inside or outside of the housing. FIGS. 2A and 2B show device configurations with magnet 2 located outside housing 11 (FIG. 2A) and inside housing 11 (FIG. 2B).

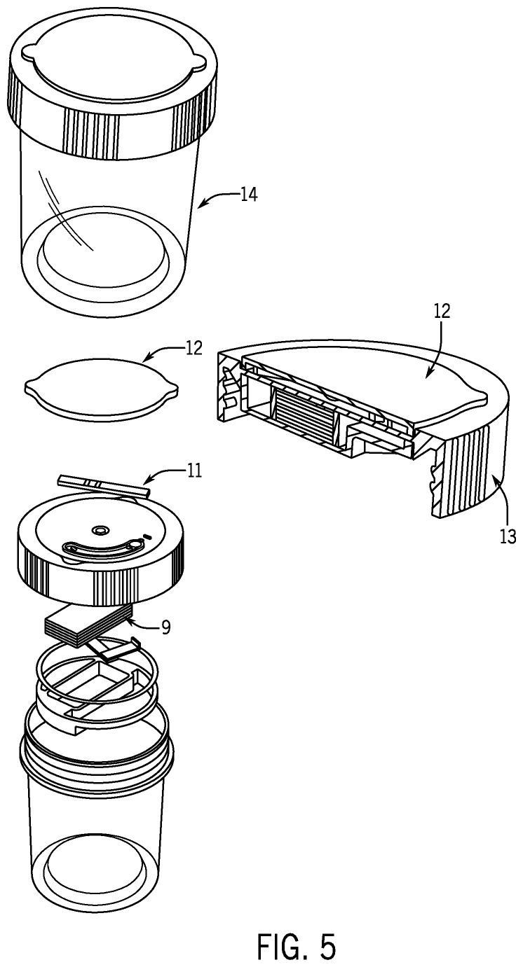

FIGS. 5-8 show an exemplary sample collection container. In some embodiments, as shown in FIGS. 5-8, the container is a cup, although other shapes of containers are specifically contemplated. In some embodiments, devices 1 (optionally comprising fluid collection components) are integrated into the lid of the container. In use, the user places a sample (e.g., urine) in the container. The container is then inverted, allowing the sample to contact the device. After sufficient time has elapsed for the sample to contact the device, the container is returned to an upright position. As described below, the magnet is then removed to allow analyte to travel to the test line.

FIG. 5 shows a close up of the lid 13 of an exemplary sample container 14 showing a lower assembly (e.g., comprising an absorbent pad 9 and assay device 1 in the lower assembly and a removable lid cover 12 in the upper assembly. The lower panel of FIG. 5 shows assay and fluid collection components in the lid assembly in a disassembled format.

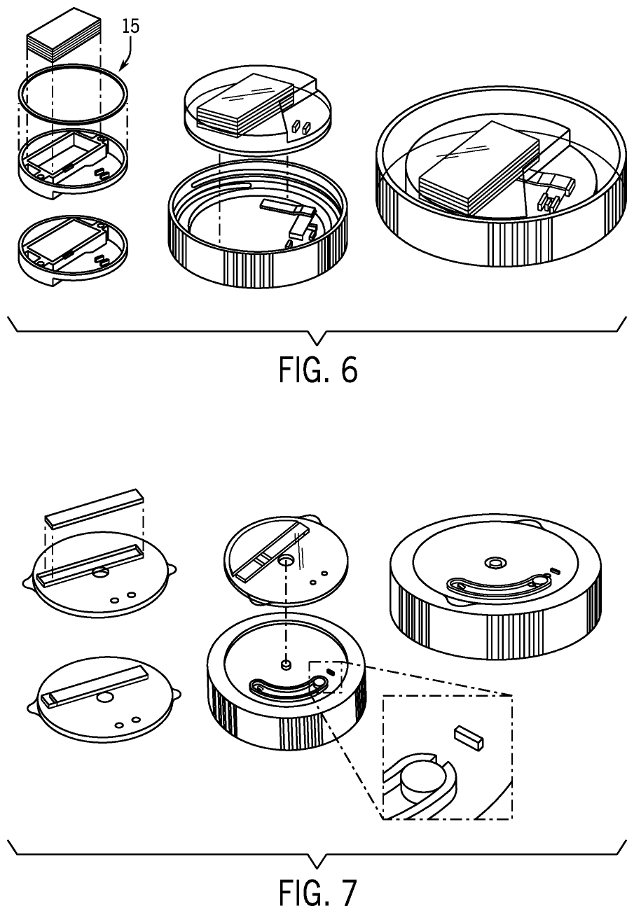

FIG. 6 shows the lower portion of the lid in an assembled (right) and disassembled (left) format. In use, after the sample has contacted the lid, the container is inverted and the lid is removed. Shown is a tape ring 15 than holds absorbed pad 9 in the plastic housing of the lid. In use, the user presses down on the pad 9 to contact sample with the sample loading component 6 and sample pad 8.

FIG. 7 shows a further embodiment of a lid assembly. An assay device is inserted into the lower assembly of the lid. A magnet sits over the beads in the assay device but is separated from the device by the lid material (e.g., plastic). When the user is ready to perform the assay, the magnet is rotated to remove the magnet from contact with the assay device and allow sample to flow to the test line.

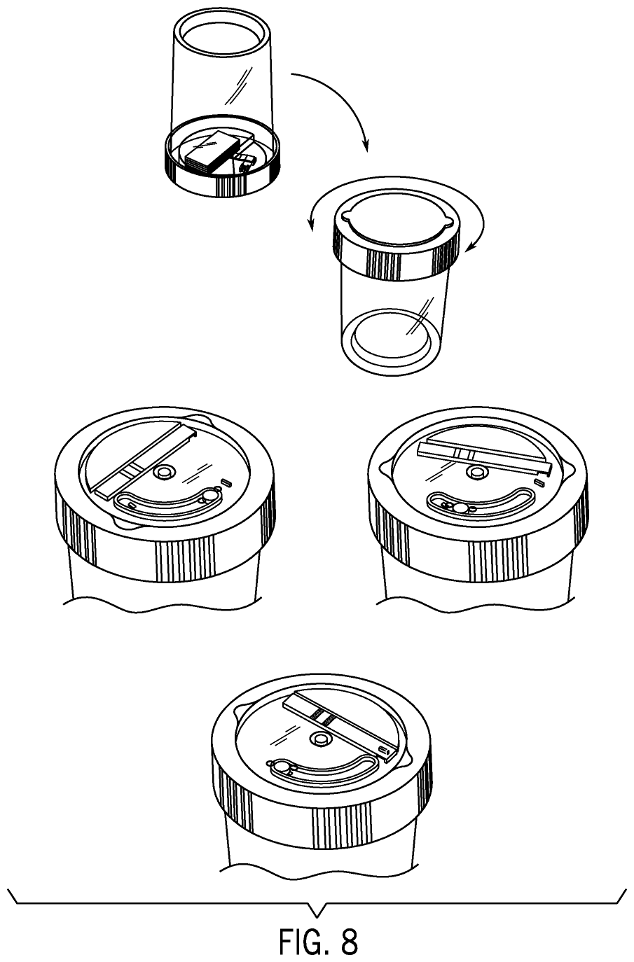

FIG. 8 shows a further view of a lid with a rotating magnet assembly. As shown, the magnet assembly allows the magnet to rotate away from contact with beads and allow beads comprising sample to flow to the test line of the assay device (shown as a lateral flow strip in FIG. 8). A portion of sample loading component 6 (e.g., glass pad) is shown exposed on the lid. When the entire lid is rotated, the assay device contacts the sample loading component and the magnet is simultaneously removed from contact with the beads.

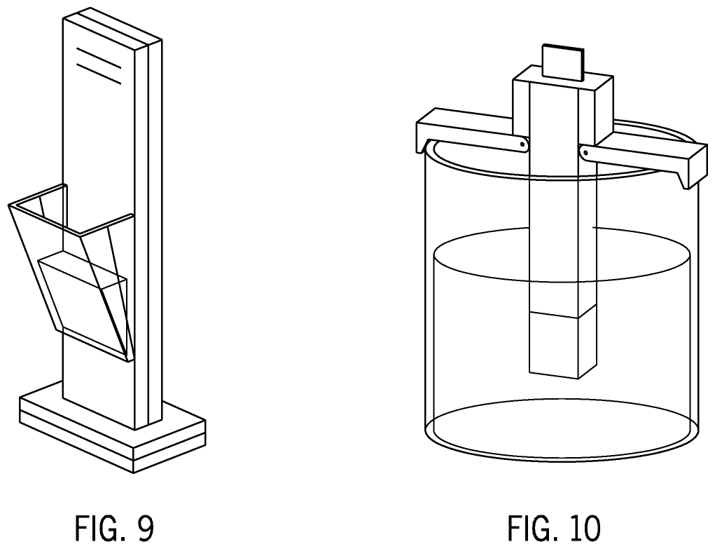

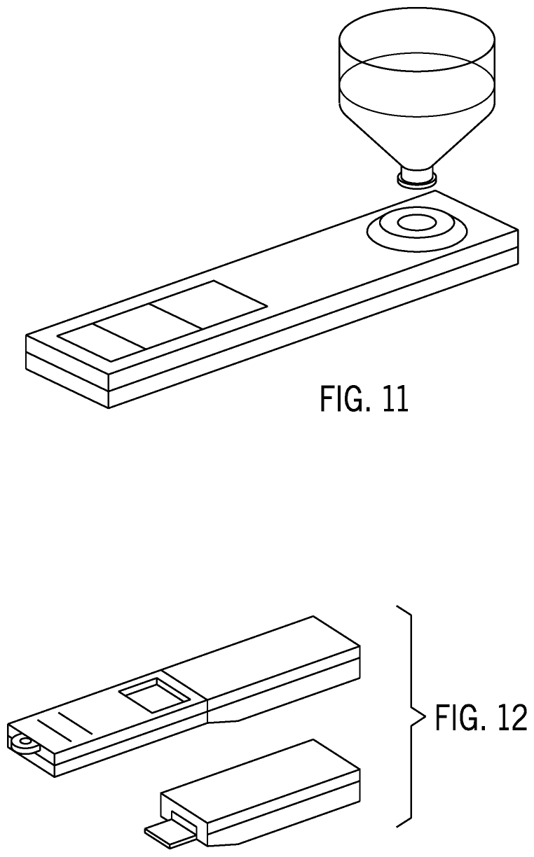

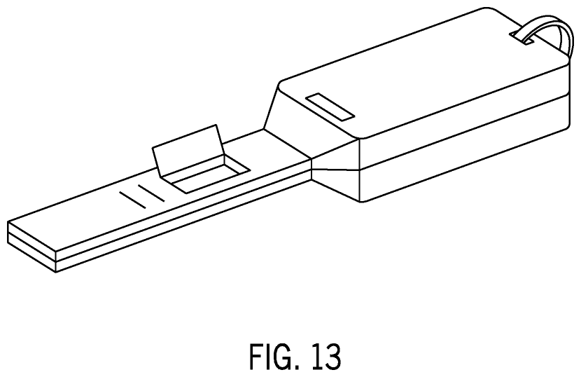

Additional housing and device configurations are shown in FIGS. 9-13. FIG. 9 shows an expandable device with removable barrier. As show in FIG. 9, a user opens a tab to obtain access to a removable barrier, which can comprises an external portion that can be pulled to remove the barrier. FIG. 10 shows the device of FIG. 9 expanded and suspended over a sample container such that a sample is absorbed into the device. The device is suspended over a sample container using expandable arms that can be folded in (e.g., for transport or analysis) or out (e.g., for sample collection). FIG. 11 shows a device with integrated sample cup or funnel for use of large volumes of sample. The sample cup or funnel is integrated as shown or removable. FIG. 12 shows a device with integrated magnet and configured for dual flow direction of sample as shown in FIG. 4. In some embodiments, the sample collection and test components can be separated as shown in the bottom portion of FIG. 12. FIG. 13 shows a further integrated device with a removable barrier tab, indicator, and readout window. In the configuration shown in FIG. 13, the device comprises a textured exterior (e.g., for easy grasping) that can be added, e.g., during the molding process.

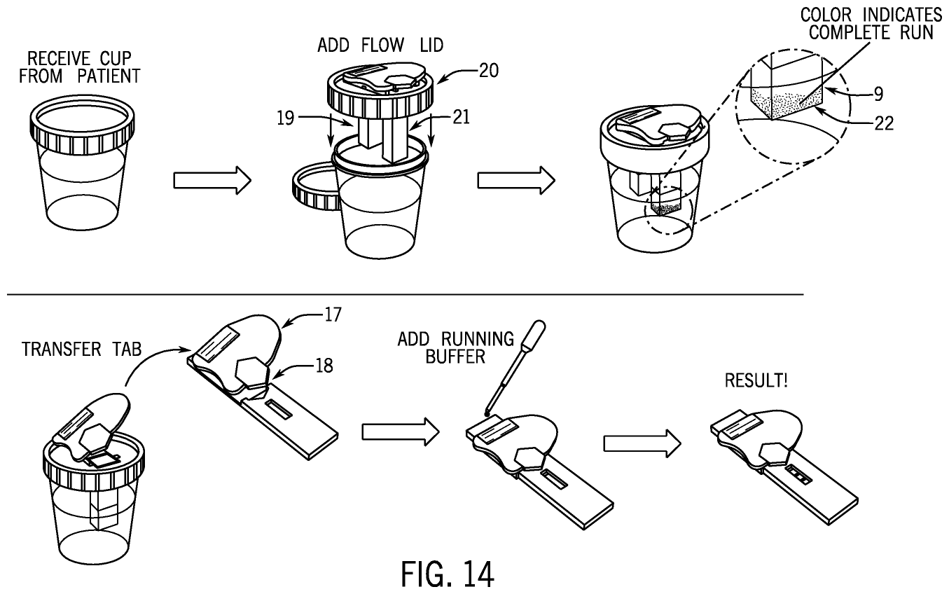

FIG. 14 shows an exemplary device with integrated lid for use with standard sample container. In use, sample (e.g., urine sample) is collected in a collection container. In some embodiments, the sample is collected by a subject, who places a standard lid on the container after collection. The lid 20 with integrated sample concentration 2 and loading component 6 is added to the cup or replaces the existing standard lid. In some embodiments, the sample collection lid 20 comprises two columns protruding from the bottom of the lid. One column (proximal column 19 in FIG. 14) has a sample loading component 6 (e.g., pad) inside and is completely sealed from the sample. The second column (distal column 21 in FIG. 14) has an absorbent pad 9 inside, and there is an access hole in the column allowing the urine to contact the absorbent pad 9. On top of the lid there is a tab 17. On the bottom of the tab is sample loading component 6 comprising particles 3 (e.g., magnetic beads) and a magnet 2 to immobilize the beads. A transfer pad (not shown) connects the sample loading component to the absorbent pad 9. When the lid is installed, sample flows through the sample loading component 6 and the analyte binds to the particles 3. The volume of flow is regulated by the size of the absorbent pad 9. When the absorbent pad is saturated, the color indicator 22 becomes visible.

After the flow is complete (color indicator is visible), the tab 17 is transferred to a test strip (e.g., LFA). The pad (in the tab) now connects sample loading component 6 (the distal end) to the LFA strip (the proximal end) via an integrated sample loading area 18 of the LFA. In some embodiments, a buffer (e.g., running buffer or elution buffer) is then added to the port on the distal end. The buffer flows through the pad on the bottom of the tab, eluting and/or carrying the analyte of the pad and into the LFA. The result is provided as a read-out on the LFA.

In some embodiments, devices are disposable or re-usable. In some embodiments, devices are provided in a system with a wash buffer for transporting sample to the test line. In some embodiments, devices and systems are designed for home use. In some embodiments, devices and systems are designed for laboratory use. In some embodiments, the use of the device is automated.

In some embodiments, devices comprise multiple test devices (e.g., specific for different analytes). For example, in some embodiments, one test device is integrated into the lid as shown in FIGS. 5-8 and a second test device is integrated with the bottom of the device. In some embodiments, such devices have openings (e.g., lids) at both the top and bottom of the device in order to allow for simultaneous testing of multiple analytes.

In use, the magnet is first placed in operable communication to secure the magnetic particles in the sample loading component. The sample is then loaded into the sample loading component of the device. In some embodiments, the magnet remains in operable communication for a period of time (e.g., several minutes) in order to allow analyte to bind to the magnetic particles. In some embodiments, the magnet is then removed from operable communication with the magnetic particles, allowing particles, bound with analyte, to travel to the test line. In some embodiments, wash buffer is then added to aid in transport of magnetic particles to the test line. In some embodiments, sample concentration and/or device operation are automated or robotically controlled. For example, in some embodiments, operation (e.g., contacting particles comprising sample with the test device) is performed autonomously (e.g., by hydrogel swelling or dissolving to trigger connection via a release of a compressed spring) or semi-autonomously (e.g. a compressed spring performs the action, but release is triggered by press of button).

The assay systems described herein find use in a variety of immunoassay applications. Examples include, but are not limited to, hormones (e.g., pregnancy or ovulation hormones), infectious disease markers or markers for environmental monitoring.

In some embodiments, the analyte to be detected is a protein, peptide, carbohydrate, lipid, small molecule, antibody, nucleic acid, virus, virus particle, drug, drug metabolite or small molecule. Specific examples include, but are not limited to, human chorionic gonadotrophin, luteinizing hormone, estrone-3-glucoronide, pregnanediol3-glucoronide, insulin, glucagon, relaxin, thyrotropin, somatotropin, gonadotropin, follicle-stimulating hormone, gastrin, bradykinin, vasopressin, polysaccharides, estrone, estradiol, cortisol, testosterone, progesterone, chenodeoxycholic acid, digoxin, cholic acid, digitoxin, deoxycholic acid, lithocholic acids; vitamins, thyroxine, triiodothyronine, histamine, serotorin, prostaglandin, drugs, drug metabolites, ferritin or CEA.

In some embodiments, immunoassays utilize antibodies to a purified protein (e.g., analyte). Such antibodies may be polyclonal or monoclonal, chimeric, humanized, single chain or Fab fragments, which may be labeled or unlabeled, all of which may be produced by using well known procedures and standard laboratory practices. See, e.g., Burns, ed., Immunochemical Protocols, 3^{rd} ed., Humana Press (2005); Harlow and Lane, Antibodies: A Laboratory Manual, Cold Spring Harbor Laboratory (1988); Kozbor et al., Immunology Today 4: 72 (1983); Köhler and Milstein, Nature 256: 495 (1975). In some embodiments, commercially available antibodies are utilized.

The devices and methods of the present invention are suitable for use with a variety of sample types. Exemplary sample types include, but are not limited to, blood, serum, nasal fluid, urine, sweat, plasma, semen, cerebrospinal fluid, tears, pus, amniotic fluid, saliva, lung aspirate, gastrointestinal contents, vaginal discharge, urethral discharge, chorionic villi specimens, skin epithelials, genitalia epithelials, gum epithelials, throat epithelials, hair or sputum.

In some embodiments, kits, systems and/or devices of the present invention are shipped containing all components necessary, sufficient or useful to perform immunoassays. In other embodiments, additional reaction components are supplied in separate vessels packaged together into a kit.

Any of these compositions, alone or in combination with other compositions disclosed herein or well known in the art, may be provided in the form of a kit. Kits may further comprise appropriate controls and/or detection reagents. Any one or more reagents that find use in any of the methods described herein may be provided in the kit.

An exemplary system for tested for the detection of analyte associated with infectious disease agents. The results indicated that devices of embodiments of the present disclosure exhibited faster detection from time of infection (e.g., 15 days compared to 20-28 days with existing systems), lower limit of detection (e.g., 10 particles/ml), and a 100-fold increase in sensitivity as a low cost. In addition, the devices described herein with amendable for use with a wide range of sample collection systems (e.g., nasal swab).

The foregoing description of illustrative embodiments of the disclosure has been presented for purposes of illustration and of description. It is not intended to be exhaustive or to limit the disclosure to the precise form disclosed, and modifications and variations are possible in light of the above teachings or may be acquired from practice of the disclosure. The embodiments were chosen and described in order to explain the principles of the disclosure and as practical applications of the disclosure to enable one skilled in the art to utilize the disclosure in various embodiments and with various modifications as suited to the particular use contemplated. It is intended that the scope of the disclosure be defined by the claims appended hereto and their equivalents.

What is claimed is:

1. A sample collection and analysis device, comprising:
a) a sample container; b) a lid, wherein said lid comprises i) a sample concentration component; ii) a sample loading component configured to integrate into a test device; iii) a fluid handling component comprising a sample pad in operable communication with said sample loading component and a plurality of absorbent pads; and iv) a test device; and c) a fluid transfer component configured to transport a sample from said sample container to said lid.

2. The device of claim 1, wherein said fluid transfer component is in fluid communication with said absorbent pads or is said absorbent pads.

3. The device of claim 1, wherein said fluid transfer component is said sample container.

4. The device of claim 1, wherein said sample loading component and/or said fluid handling pad are in fluid communication with said sample container.

5. The device of claim 1, wherein said fluid handling component and said absorbent pad are connected by a transfer pad.

6. The device of claim 1, wherein said test device is a lateral flow assay.

7. The device of claim 5, wherein said test device is in fluid contact with said sample loading component.

8. The device of claim 5, wherein said test device is not in fluid contact with said sample loading component.

9. The device of claim 8, wherein said test device is separated from said sample loading component by a physical barrier.

10. The device of claim 1, wherein said sample loading component is removable.

11. The device of claim 10, wherein said sample loading component is on a removable tab.

12. The device of claim 10, wherein said removable tab is configured to integrate with said test device.

13. A sample collection and analysis device, comprising:
a) a sample container; b) a lid, wherein said lid comprises i) a first column protruding from said lid and configured to contact a sample in said sample container, and comprising a sample concentration component; ii) a second column protruding from said lid and configured to contact a sample in said sample container, and comprising a fluid handling component comprising a sample pad and a plurality of absorbent pads; and iii) a sample loading component configured to integrate into a test device.

14. The device of claim 13, wherein said first and second columns are in fluid communication.

15. A method of detecting an analyte in a sample, comprising:
A) introducing a sample into a sample collection and analysis device, comprising:
a) a sample container; b) a lid, wherein said lid comprises i) a sample concentration component; ii) a sample loading component configured to integrate into a test device; iii) a fluid handling component comprising a sample pad in operable communication with said sample loading component and a plurality of absorbent pads; and iv) a test device; and c) a fluid transfer component configured to transport a sample from said sample container to said lid such that said sample is concentrated in said sample concentration component; and
B) contacting said concentrated sample with a test device to detect said analyte.

16. The method of claim 16, wherein said introducing comprises inverting said sample container.

17. The method of claim 15, wherein said introducing comprises allowing sample to flow up through columns protruding from the bottom of said lid.

18. The method of claim 15, wherein said test device is integrated into said sample collection device.

19. The method of claim 15, wherein said test device is not integrated into said sample collection device.

20. The method of claim 19, wherein said fluid transfer component is an absorbent pad.
